(19) United States
(12) Patent Application Publication
(10) Pub. No.: US 2005/0197323 A1
(43) Pub. Date: Sep. 8, 2005

(54) Use of 5-HT3 receptor antagonists for the treatment of inflammations of the respiratory tract

(76) Inventors: Lothar Farber, Heroldsberg (DE); Wolfgang Muller, Binningen (CH); Christian Stratz, Freiburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/122,809

(22) Filed: May 5, 2005

Related U.S. Application Data
(63) Continuation of application No. 10/048,512, filed on May 23, 2002, now abandoned.

(30) Foreign Application Priority Data
Aug. 4, 1999 (GB) 9918425.1

(51) Int. Cl. A61K 31/573; A61K 31/4178

(52) U.S. Cl. 514/171; 514/397

(57) ABSTRACT

It has been found that compounds having 5-HT₃ (serotonin M) receptor antagonist activity can be used for (the manufacture of medicaments for) treating inflammations of the respiratory tract including the larynx, the trachea, or especially the bronchi and the lung, especially for treating obstructive pulmonary/bronchial diseases, or laryngospams (Spasmus glottides).

The present invention relates to a new use, in particular a new pharmaceutical use for compounds having 5-HT₃ (serotonin M) receptor, in particular specific 5-HT₃ receptor, antagonist activity, for a new treatment of inflammations of the respiratory tract.

Specifically, the present invention relates to the treatments defined below.

The 5-HT₃-receptor antagonists comprise a defined and recognised class of pharmaceutically active compounds well known in the art and characterised, as their name implies, by their pharmacological activity. Various 5-HT₃ receptor antagonist compounds are commercially available and clinically applied, e.g. in the treatment of emesis.

In accordance with the present invention it has now surprisingly been found that 5-HT₃ receptor antagonists are useful for the treatment of inflammatory diseases of the respiratory tract, especially obstructive pulmonary/bronchial diseases, or laryngospasm.

This is surprising in that the 5-HT₃ receptor antagonists are effective even alone.

Any 5-HT₃ receptor antagonist can be used in accordance with the invention. Preferred 5-HT₃ receptor antagonists which may be employed in accordance with the present invention are:
A) Ondansetron [1,2,3,9-tetrahydro-9-methyl[(2-methyl-1H-imidazol-1-yl]methyl]-4H-carbazol-4-one (cf. Merck Index, twelfth edition, item 6979);
B) Granisetron [endo-1-methyl-N-(9-methyl-9-aza-bicyclo[3.3.1]non-3-yl)-1H-imidazole-3-carboxamide: (cf. loc. cit, item 4557); and
C) Dolasetron [1H-indole-3-carboxylic add (2α,6α,8α,9αβ)-octahydroxo-3-oxo-2,6 methano-2H-quinolizin-8-yl ester] (cf. k cit, item 3471). Particular 5-HT₃ receptor antagonists which may be employed in accordance with the present invention are those of the formula 1 as defined in European Patent Publication 189002 B1, the contents of which are incorporated herein by reference, in particular the compound:
D) indol-3-yl-carboxylic acid-endo-8-methyl-8-aza-bicyclo[3,2,1]-oct-3-yl-ester, also known as Tropisetron. (cf. loc.cit., item 9914).

Further 5-HT₃ receptor antagonists which may be used preferably in accordance with the present invention are:
E) 4,5,6,7-tetrahydro-5-[(1-methyl-indol-3-yl)carbonyl]benzimidazole (see also Ramosetron, see U.S. Pat. No. 5,344,927);
F) (+)-10-methyl-7-(5-methyl-1H-imidazol-4-ylmethyl)-6,7,8,9-tetrahydropyrido[1,2-a]indol-6-one (see also Fabesetron, EP 0 361 317); and
G) [N-(1-ethyl-2-imidazolin-2-ylmethyl)-2-methoxy-4-amino-5-chlorobenzamide (see also Lintopride—Chem.-Abstr.-No. 10742963-0).

A further 5-HT₃ receptor antagonists which may be used preferably in accordance with the present invention is
(H) 2,3,4,5-tetrahydro-5-methyl-2-[(5-methyl-1H-imidazol-4-yl)methyl]-1H-pyrido[4,3-b]indol-1-one (see also Alosetron, EP 0 306 323).

5-HT₃-receptor antagonists may be employed in accordance with the invention in free or in pharmaceutically acceptable salt form, e.g. as known in the art, for example, in the case of compounds A) to D) above in pharmaceutically acceptable acid addition salt form, for example, in the case of compound A) the hydrochloride dihydrate; corn pound B) the hydrochloride; compound C) the mesylate; and compound D) the mono hydrochloride. References to 5-HT₃ receptor antagonist collectively or individually throughout the present specification and claims are accordingly to be understood as embracing both free compounds and such pharmaceutically acceptable salt forms, e.g. as clinically employed, and further also solvates, e.g. hydrates, or specific crystal forms of any of these compounds or salts.

For use in accordance with the present invention tropisteron (especially in the formulation called NAVOBAN®) is most preferred.

In accordance with the present invention it has now surprisingly been found that 5-HT₃-receptor antagonists are useful for the treatment of diseases, processes, conditions and events in the respiratory tract, especially the lower respiratory tract including the larynx, the trachea, or especially the bronchi and the lung; preferably of pulmonary/bronchial obstructive diseases, processes, conditions or events, or of laryngospasm (Spasmus glottidis).

“Treatment” as used herein includes use for the alleviation, amelioration or control of the diseases, processes, conditions or events mentioned above and below. The term includes therapeutic, symptomatic or in a broader sense also prophylactic treatment. It also includes intervention for the alleviation, amelioration or control of the sequelae or symptoms of any one or more of these diseases, for example degeneration (e.g. of cells, epithelia or tissues), such as fibrosis; swelling, especially oedema, effusion, e.g. exudation, emphysema or pain, and most especially dyspnea. In this context the term “treatment” is further to be understood as embracing use to reverse, restrict or control progression of any specified disease, process, condition event or the like, including use for disease modifying effect. The term “treatment” preferably encompasses the alleviation, amelioration or control (including termporal or permanent removal) of at least one further sequela or symptom in addition to dyspnea, such as swelling, effusion, oedema, emphysema, fibrosis or other degeneration, but especially dyspnea, e.g. due to bronchoconstriction, oedema, exudation, swelling, emphysema or the like.

Therapeutic and prophylactic efficacy in the treatment of pulmonary/bronchial obstructive diseases will be evidenced by improvement in lung function or diminished airway hyperreactivity, a reduced requirement for other symptomatic therapy, i.e. therapy for or intended to restrict or abort a symptomatic attack when it occurs, for ex ample anti-inflammatory (e.g. corticosteroid) or bronchodilatatory.

The present invention is in particular applicable to the treatment of:
(I) pulmonary/bronchial obstructive diseases especially
(1) Chronic Obstructive Pulmonary Disease (COPD);
(2) Adult Respiratory Distress Syndrome (ARDS); or, in a broader sense of the invention, also
(3) bronchitis of whatever type or genesis including, e.g., chronic, acute, arachidic, catarrhal, croupous, chronic or phthinoid bronchitis; or
(4) pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, ptilosis, siderosis, silicosis, tabacosis and byssinosis;
(II) or (in a broader aspect of the invention)
(5) laryngospasm;
(6) pulmonary vasculitis;
(7) pulmonary granulomatosis; or
(8) extrinsic allergic alveolitis;
   or any combination of more than one of the diseases mentioned under (I) and (II).

Preferably, the invention relates to the treatment of any of the diseases mentioned above under (I) or under (II). More preferably, the invention relates to the treatment of any one of the diseases (1), (2), (3), (4), (5), (6), (7), and (8) mentioned above.

In the case that an infection (e.g. bacterial, fungal, protozoal, parasitic or viral infection) is present, the treatment with a 5-HT₃ receptor antagonist is not to be understood to replace therapy of the underlying infection with appropriate measures (including medication, e.g. with chemotherapeutics, or other therapeutic measures); bearing in mind that treatment with a 5-HT₃ receptor antagonist may still be necessary e.g. in cases of otherwise life-threatening dyspnoe.

In a further aspect it has been found in accordance with the present invention that 5-HT₃ receptor antagonists are useful as replacement therapy for glucocorticosteroid, e.g. cortisone or the like, therapy; for example for use in any means of treat ment as hereinbefore and hereinafter set forth, e.g. the diseases mentioned under (I) and (II) hereinabove.

The term “replacement therapy” as used herein is to be understood as embracing both use “as full replacement”, i.e. use instead of glucocorticosterold therapy, as well as use “as partial replacement” for glucocorticosteroid therapy, i.e. for administration together with glucocorticosteroid therapy or as a means of reducing glucocorticosteroid dosage or to achieve a glucocorticosteroid sparing effect.

The present invention accordingly provides:
(i) A method of treating any process, condition, event, or disease as hereinbefore set forth, in a subject in need thereof, which method comprises administering an effective amount of a 5-HT₃ receptor antagonist;
   (ii) A method of providing replacement therapy for glucocorticosteroid therapy in a subject receiving such glucocorticosteroid therapy for or in the treatment of any process, condition, event or disease as hereinbefore set forth, which process comprises administering to said subject an effective amount, e.g. a glucocorticosteroid sparing amount, of a 5-HT₃-receptor antagonist; as well as
(iii) A method of treating any process, condition, event or disease as hereinbefore set forth, in a subject in need thereof, which method comprises administering an effective amount of a 5-HT₃ receptor antagonist together with a glucocorticosteroid.

Where the term “glucositeroid” is used, this means an anti-inflammatory glucosteroid, especially a corticosteroid, such as Cortisone.

Other therapeutics that can be complemented or replaced with 5-HT₃ receptor antagonist treatment include, but are not limited to, brochospasmolytics or inhibitors of mediator release, e.g. cromoglicic acid. These can be added in place of the term “glucosteroid” in any of the definitions hereinbefore or hereinafter, in a broader aspect of the invention.

Where co-administration is practiced as under (Cii) above the drug substances, i.e. 5-HT₃ receptor antagonist and glucocorticosteroid may be administered sequentially or simultaneously or substantially simultaneously, e.g. employing a fixed combination dosage form.

In further aspects the present invention also provides:
(iv) A 5-HT₃ receptor antagonist for use in, or for use in the manufacture of a pharmaceutical composition for use in; or the use of a pharmaceutical composition comprising a 5-HT₃ receptor antagonist:
   a) the treatment of any process, condition, event or disease as hereinbefore set forth;
   b) as replacement therapy for glucocorticosteroid therapy in the treatment of any process, condition, event or disease as hereinbefore set forth; or
   c) for co-administration together with a glucocorticosteroid in the treatment of any process, condition, event or disease as hereinbefore set forth; as well as
(v) A pharmaceutical dosage form comprising a 5-HT₃ receptor antagonist together with a glucocorticosteroid, especially for the treatment of any process, condition, event or disease as hereinbefore set forth.

Where under (i) to (v) the term “any process, condition, event or disease” is used, this term preferably relates to the diseases mentioned under (I) and (II) above, especially as defined above as being preferred.

Dosage forms, e.g. In accordance with v above, are to be understood as including both fixed-unit-dosage forms, e.g. liquid formulations or solid formulations that have to be completed by the addition of water, physiologicyl saline or the like, comprising both active ingredients together with appropriate pharmaceutically acceptable diluents or carriers, as well as twin delivery systems, packages or the like comprising both active ingredients separately or in separate dosage form, for concommitant or sequential administration.

The 5-HT₃ receptor antagonists are preferably used in well-known liquid formulations.

Utility of 5-HT₃ receptor antagonists in accordance with the present invention can be demonstrated in clinical trials carried in accordance with standard techniques and methodologies, for example as follows:

The following example is for illustrative purposes and is not intended to diminish the scope of the present invention. Instead of tropisetron, any other 5-HT₃-antagonist, or a pharmaceutically acceptable salt thereof, solvate, e.g. hydrate, or crystalline form thereof, especially selected from the group consisting of Ondansetron, Granisetron, Dolasetron, Ramosetron, Fabesetron, Lintopride and Alosetron, can be used.

In the following example, tropisetron is administered in the standard formulation of the trademark Navoban® which is available in ampoules that contain 2 mg (or 5 mg) of the active substance, tropisetron.

### EXAMPLE

Male patient, born in 1929, suffering from COPD for 10 years. Immediately before administration of 2 mg Navoban®, the patient in the Tiffeneau test (one-second forced expiratory volume—determines the maximal expiration speed in one second in l/sec) shows a maximal forced expiratory volume of 0.45 l/sec, 1 h after treatment of 0.5 l/sec, 4 h after treatment of 0.75 l/sec, and 24 h after treatment of 0.55 l/sec. In parallel, the vital capacity (maximal expirable volume after maximal inspiration, wit time limitation; given in I) changes from 1.3 l immediately before administration to 1.5 l 1 h after treatment, 1.8 l 4 h after treatment and 1.4 l 24 h after treatment. This indicates ameliorated performance both in the Tiffeneau test and the vital capacity.

Equivalent results as in the preceding example are obtainable in equivalent or comparable trials with patients exhibiting similar symptomatology employing 5-HT₃-receptor antagonists other than tropisetron, for example using any of the 5-HT₃-receptor antagonists A) through C) or E) through H) hereinbefore recited at comparable, e.g. conventional clinical, dose as known in the art. Similar results are also achievable employing 5-HT₃-receptor antagonists, e.g. tropisetron at doses of the order of 2 mg/day p.o. or by injection (i.m., iv.v or i.d.) or by other ways of administration in clinical trials involving subjects exhibiting other diseases as defined above.

The trial conducted as described above or analogously is demonstrative of long lasting and disease modifying effects in conditions herein described as well as symptomatic and gluco corticosteroid replacement effect for 5-HT₃ receptor antagonists.

For use in accordance with the present invention the appropriate dosage will, of course, vary depending on for example the particular 5-HT₃ receptor antagonist employed the mode of administration and the nature and severity of the condition to be treated as well as the specific condition to be treated. In general an indicated single, e.g. daily, dosage will be in the range usually employed for known indications such as emesis and will typically be from about 0.05 to about 50 mg per day, more preferably around 1 to 10 mg per day, conveniently administered once or in divided doses up to four times a day or in sustained release form, or used repeatedly after longer intervals, e.g. after some days or weeks, e.g. after 2 days to 4 weeks. In the case of tropisetron an appropriate dosa ge for administration, e.g. by injection, for example for i.v. application or i.m. injection, will be of the order of 2 mg per day or 5 mg per day, administered once, sequentially over a sequence of 2 to 20 days or at intervals of 2 to 5 days to 2 days to 2 weeks.

For use in accordance with the invention, 5-HT₃ receptor antagonists may be administered by any conventional route in particular enterally, preferably orally, e.g. in the form of tablets or capsules, or lozenges for e.g. buccal administation; or more preferably intra-nasally or especially by inhalation, e.g. in the form of aerosoles or powders; or rectally, e.g. in the form of suppositories or enemation; or most preferably parenterally, e.g. in the form of solutions or suspensions for injection or infusion, e.g. by subcutaneous, intraperitoneal, intradermal or intramuscular injection for systemic administration.

In addition, transdermal administration may be considered, e.g. by use of gels, creams or ointments or the like, or preferably by transdermal patches. Dosages for such forms will be of the order or slightly higher than those used on administration by injection.

Suitable formulations for use in accordance with the present invention will include any of those as known and commercially available and clinically employed in the art, for example the commerically available formulations.

In more general terms, the formulations can be selected from formulations such as those described in standard text books, e.g. The United States Pharmacopoieia (e.g. the 1970 version by The United States Pharmacopoeia Convention, Mack Printing Company, 18.th edition, 1970; or 22nd edition, 1989), or further “Pharmazeutische Technologie”, R. H. Müller and G. E. Hildebrand, Wissenschaftliche Verlagsgesellschaft mbH, Stuttgart 1998, or “Pharmazeutische Technologie”, Heinz Sucker, Peter Fusch and Peter Speiser (eds.), 2nd edition, Georg Thieme Verlag, Stuttgart/New York 1991; and with standard excipients, e.g. those mentioned in the Handbook of Pharmaceutical Excipients (Ainley Wade and Paul J. Weller, eds.), Second Edition, The Pharmaceuticals Press, London 1994. The preparation of the formulations is also possible in accordance with standard textbooks, such as those just mentioned.

1-11. (canceled)

12. A method of treating an inflammatory disease of the respiratory tract in a subject in need thereof, which method comprises administering to said subject an effective amount of a 5-HT₃ receptor antagonist.

13. A method according to claim 12 wherein the 5-HT₃ antagonist is administered by injection.

14. A method according to claim 12 where the 5-HT₃ receptor antagonist is selected from the group consisting of ondansetron, granisetron, dolasetron, tropisetron, ramosetron, fabesetron, lintopride and alosetron, in free form or as a pharmaceutically acceptable salt thereof.

15. A method of providing replacement therapy for glucocorticosteroid therapy in a subject receiving such glucocorticosteroid therapy, which method comprises administering to said subject a glucocorticosteroid sparing amount of a 5-HT₃ receptor antagonist.

16. A method according to claim 12 comprising administering a glucocorticosteroid together with said 5-HT₃ receptor antagonist.

17. A pharmaceutical dosage form comprising a 5-HT₃ receptor antagonist together with an anti-inflammatory glucocorticosteroid.

18. A method according to claim 14 wherein the 5-HT₃ receptor antagonist is tropisetron.

19. A method according to claim 12 wherein said inflammatory disease is selected from a pulmonary/bronchial obstructive disease.

20. A method according to claim 19 wherein said inflammatory disease is selected from the group consisting of
a) Chronic Obstructive Pulmonary Disease (COPD);
b) Adult Respiratory Distress Syndrome (ARDS);
c) bronchitis; or
pneumoconiosis.

21. A method according to claim 12 wherein said inflammatory disease is selected from the group consisting of a) laryngospasm;
b) pulmonary vasculitis;
c) pulmonary granulomatosis; or
d) extrinsic allergic alveolitis;
or any combination thereof.

22. A method according to claim 20 wherein said disease is COPD.

23. A method according to claim 16 wherein said 5-HT₃ receptor antagonist is selected from the group consisting of ondansetron, granisetron, dolasetron, tropisetron, ramosetron, fabesetron, lintopride and alosetron, in free form or as a pharmaceutically acceptable salt thereof.

24. A method according to claim 23 wherein said 5-HT₃ receptor antagonist is tropisetron.

25. A method according to claim 16 wherein said glucocorticosteroid is a corticosteroid.

26. A method according to claim 24 wherein the glucocorticosteroid is a corticosteroid.
